(19) United States
(12) Patent Application Publication
(10) Pub. No.: US 2004/0126823 A1
(43) Pub. Date: Jul. 1, 2004

(54) Modulation of prostaglandin synthesis and cancer growth

(76) Inventors: Philip N. Tsichlis, Willow Grove, PA (US); Calin Dan Dumitru, Philadelphia, PA (US); Aristides G. Eliopoulos, Birmingham (GB)

Correspondence Address: REED SMITH LLP, 2500 ONE LIBERTY PLACE, 1650 MARKET STREET, PHILADELPHIA, PA, 19103, US

(21) Appl. No.: 10/453,806

(22) Filed: Jun. 2, 2003

Related U.S. Application Data
(60) Provisional application No. 60/384,901, filed on May 31, 2002.

(51) Int. Cl. G01N 33/574

(52) U.S. Cl. 435/7.23

(57) ABSTRACT

The present invention discloses novel methods to identify compounds potentially useful for the treatment and prevention of inflammation and/or cancer in animals including mammals. It disclose that Tpl2 is required for tumor induction by Akt and Tpl2 is required for the induction of cyclo-oxygenase-2 (COX-2) and prostaglandin synthesis and provides methods to identify compounds that modulate interactions between Tpl-2 and COX-2 or interactions between Tpl-2 and Akt. The present invention also discloses a transgenic Tpl2−/− mouse encoding Akt where the mouse is characterized by its ability to show delayed tumor induction by comparison with a transgenic Tpl2+/+ mouse expressing the Akt and method of treating cancers in animals.

This application claims the benefit of U.S. Provisional Application No. 60/384,901, filed May 31, 2002, and the text of application 60/384,901 is incorporated by reference in its entirety herewith.

### FIELD OF THE INVENTION

The present invention relates to novel methods to identify compounds potentially useful for the treatment and prevention of cancer and/or inflammation in animals including mammals as well as pharmaceutical compositions containing such compounds. Specifically it relates to methods for identifying compounds that modulate interactions between Tpl-2 and COX-2 or interactions between Tpl-2 and Akt in cells and/or animals. Further, specifically it relates to transgenic Tpl-2 knockout animals expressiong Akt.

### BACKGROUND OF THE INVENTION

Both cancer and inflammation in animals are known to involve interaction of a number of biochemical molecules, complex biochemical cycles and signalling pathways. Biochemical cycles have many intermediate steps and intermediate biochemical molecules.

The cyclooxygenase cycle is known to be associated with both cancer and inflammation. One of the intermediates in the cyclooxygenase cycle that has been extensively studied is prostaglandin H2 synthase, which has two forms: COX-1 and COX-2. Of these two forms, COX-1 is known as a housekeeping biochemical molecule which helps generate substances that protect the stomach. The well-known inhibitor of COX-1 is aspirin. Because it inhibits a substance that protects the stomach, often it has gastrointestinal side effects. Recently, compounds including compounds isolated herbal extracts have become available that selectively inhibit COX-2 enzymes over COX-1 enzymes. COX-2 enzymes regulate pain, inflammation and fever, i.e. inflammatory mechanisms. COX-2 is also known to contribute to the progression of cancer. Further, it is known in the art that, cancer can trigger long-term inflammatory mechanisms. For inflammation, COX inhibitors are commonly used but these inhibitors are known to have considerable side effects when taken on chronic basis.

Similarly, Akt gene is another well known biochemical molecule that plays key role in the induction and proliferation of cancer cells. A number of drugs including antisense oligo nucleotides or sequences for RNAi are being developed to interfere with genes like Akt and thereby preventing cancer growth induced by them.

The present invention proposes to selectively modify a biochemical cycle so as not to destroy overall body function but affects the functions of COX-2 and Akt biochemical molecules in causing cancer and/or inflammation.

### SUMMARY OF THE INVENTION

The present invention involves, among other things, a novel a method for screening for compounds that interfere with Tpl2 dependent regulation of the expression of COX-2 or Akt induced tumors and a method for treating a condition associated with the expression of these genes by administering a compound identified in the screening method.

Specifically, the present invention discloses, among other things, a novel in vitro and/or in vivo methods for selecting compounds for their ability to treat and prevent cancer and inflammation safely. The compounds are selected for their ability to selectively modify Tpl2 so as to interefere with the functions of COX-2 and/or Akt biochemical molecules. Thus, in one aspect, the present invention is a method for selecting compounds that can be used to treat and prevent cancer and inflammation. The compounds so identified can have less severe side effects than those attributable to COX inhibitors and other non-specific interactions associated with conventional chemotherapeutics. The compounds of interest can be tested by exposing cancer cells (e.g., colon cancer cells or mammary adenocarcinoma cells) to the Tpl-2 inhibiting compounds, and if such a compound reduces the induction of both PGE2 and its regulatory enzyme COX-2, the compound is then further evaluated (e.g., in vitro or in vivo animal or human testing models or trials) for its other properties (e.g., its ability to induce apoptosis or reduce inflammatory responses in vitro and/or in vivo). Likewise, the compounds of interest can be tested by exposing cells expressing MyrAkt/Tpl2+/+ (e.g., macrophages isolated from transgenic mice expressing MyrAkt) to the Tpl-2 inhibiting compounds, and if such a compound reduces or blocks oncogenic signals transduced by Akt, the compound is then further evaluated (e.g., in vitro or in vivo animal or human testing models or trials) for its other properties (e.g., its ability to induce apoptosis in vitro and/or in vivo).

Thus, one embodiment of the novel method of this invention is evaluating whether a compound interferes with Tpl-2 expression, and reduces the induction of PGE2 and/or its regulatory enzyme COX-2. Another embodiment of the novel screening method of this invention is evaluating whether a compound interferes with Tpl-2 expression, blocks oncogenic signals transduced by Akt. Compounds successfully evaluated in such fashions have application as antineoplastics and/or anti-inflammatories.

By selecting compounds in this fashion, potentially beneficial and improved compounds for treating cancer and/or inflammation can be identified more rapidly and with greater precision than possible in the past for the purposes of developing pharmaceutical compositions and therapeutically treating cancer and/or inflammation.

In another aspect of the invention, A transgenic Tpl2−/− animal expressing MyrAkt is disclosed. The animal is can be phenotypically characterized, for example, by its ability to show delayed tumor induction by comparison with a transgenic Tpl2+/+ mouse expressing the MyrAkt.

In still another aspect of the invention, a method of screening for biologically active compounds that modulate tumor induction associated with the interaction of Tpl-2 and Akt is provided. The method includes administering a candidate compound to or combining a candidate compound with a transgenic Tpl2+/+ mouse and Tpl2−/−mouse, both comprising a transgenic nucleotide sequence encoding bioactive Akt operably linked to a promoter and stably integrated into the genome of each mouse, wherein said nucleotide sequence is expressed and wherein said expression results in delayed tumor induction in the transgenic Tpl2−/−mouse; and determining the effect of the compound upon tumor induction in the transgenic mice; and selecting the compound that controls or delays tumor induction in the Tpl2+/+ mouse as seen in the Tpl2−/−mouse. The bioactive Akt means that the encoded Akt is oncogenic. Different Akt oncogenic sequences are known in the art (v-akt, which is constitutively active, is one such example. Other examples are c-akt with a Src-derived myristoylation signal and Akt mutant, such as AktE40K).

In still another aspect of the invention, a method of screening for biologically active compounds that modulate a pathology associated with cancer, wherein the parthology is promoted by the interaction of Tpl-2 and Akt is provided. The method includes administering a candidate compound to a transgenic mouse comprising a transgenic nucleotide sequence encoding bioactive Akt operably linked to a promoter and stably integrated into the genome of the mouse, wherein said nucleotide sequence is expressed and wherein said expression results in tumor induction; and determining the effect of the compound upon the pathology promoted by the interaction of Tpl-2 and Akt.

The compound may be administered in a variety of ways including orally, parenterally, subcutaneously, intramuscularly or intravascularly or topically or by viral infection. Depending on the manner of introduction, the compounds can be formulated in a variety of ways.

This invention also includes pharmaceutical compositions containing such compounds, as well as therapeutic methods involving such compounds. All animal methods of treatment or prevention described herein are preferably applied to mammals, most preferably to humans. Further benefits will be apparent from the following detailed description.

### BRIEF DESCRIPTION OF THE FIGURES

FIG. 1 shows that LPS-treated Tpl2−/− macrophages secrete reduced levels of PGE2 as compared to Tpl2−/− macrophages.

FIG. 2 shows data on the effect of Tpl2 on COX-2 expression. (A) COX-2 induction in LPS-stimulated macrophages. (B) Exogenous Tpl2 expression in RAW264.7 macrophages

FIG. 3 shows data on the phosphorylation and DNA binding activity of CREB in LPSstimulated macrophages. (A) Kinetics of ERK phosphorylation in cell lysates from Tpl2+/+ and Tpl2−/− macrophages stimulated with LPS. (B) Kinetics of CREB/ATFI phosphorylation in cell lysates from Tpl2+/+ and Tpl2−/− macrophages stimulated with LPS. (C) Induction of CREB DNA binding activity by LPS. Left panel. Nuclear extracts of primary Tpl2+/+ and Tpl2−/− macrophages treated with LPS. Right panel. Nuclear extracts from Tpl2+/+cells treated with LPS and incubated with unlabelled wild type (wt) or mutant (mt) probe.

FIG. 4 shows data on the effect of Tpl2 transduced LPS signals on the phosphorylation status and activation of the protein kinases p90Rsk and Msk. (A) illustrates the phosphorylation status of p90Rsk in LPS-stimulated Tpl2−/− macrophages (samples probed with antibodies specific for the phosphorylated form of p90Rsk (upper panel) or for total p90Rsk (lower panel). (B) illustrates the activation of p90Rsk by LPS, in Tpl2+/+ and Tpl2−/− macrophages, or Tpl2+/+ macrophages pre-treated with PD98059 or Ro318220 and harvested at the indicated time points. (C) illustrates the phosphorylation status of Msk1 in LPS-stimulated Tpl2−/− macrophages (samples were probed with antibodies specific for the phosphorylated form of Msk1 (upper panel) or for total Msk1 (data not shown). (D) illustrates the activation of Msk1 by LPS in Tpl2+/+ and Tpl2−/− macrophages or Tpl2+/+ macrophages pre-treated with PD98059 or Ro318220 and harvested at the indicated time points.

FIG. 5 shows LPS induced expression of C/EBPP.

FIG. 6 shows a model of Tpl2-mediated COX-2 transactivation and PGE2 production in response to LPS.

FIG. 7 is a graph showing the effect of Tpl2 inactivation on the tumor induction by MyrAkt.

### DETAILED DESCRIPTION OF THE INVENTION

The present invention discloses methods to down regulating and/or blocking the effects of cancer inducing genes such as COX-2 and Akt and trasgenic animal models to screen for drugs that down regulate and/or block COX-2 and Akt effects. Specifically, it discloses that by selectively modifying a biochemical cycle associated with Tpl-2 gene it is possible to down regulate and/or block COX-2 and Akt effects.

The serine/threonine protein kinase Tpl2, also known as Cot, was identified as a target of provirus integration in MoMuLV-induced rat T cell lymphomas and MMTV-induced mammary carcinomas (1,2). Provirus insertion always occurs in the last intron of the gene, and results in the expression of a carboxy-terminally truncated, constitutively active Tpl2 kinase (1, 3, 4). When overexpressed in a variety of cell types, Tpl2 activates ERK, JNK, p38MAPK, and the transcription factors NF-AT and NF-□B (3-9). As a result, T cell lymphoma lines engineered to over-express Tpl2, constitutively express and secrete IL-2 (8). Moreover, transgenic mice expressing the truncated form of Tpl2 under the control of a T cell specific promoter develop T cell lymphoblastic lymphomas at an early age (4). The preceding data suggesting that Tpl2 is a highly oncogenic kinase in animal models were complemented recently by additional data showing that Tpl2 is overexpressed in human breast cancer and other epithelial tumors. The latter data provided evidence for the involvement of Tpl2 in the development of human cancer.

Recent studies on cells derived from Tpl2−/− mice showed that the Tpl2 kinase plays a key physiological role in LPS signaling. LPS activates all the signaling pathways that are also activated by Tpl2. However, in LPS-stimulated cells Tpl2 is required only for the activation of ERK. Because of this signaling defect, LPS-stimulated macrophages from Tpl2−/− mice are defective in the transport of the TNF-α mRNA from the nucleus to the cytoplasm, and they are impaired in the induction of TNF-α by LPS. As a result, Tpl2−/− mice are resistant to LPS/D-galactosamine-induced endotoxin shock (10).

Additional studies revealed that not only TNF-α expression, but also TNF-α signaling are defective in Tpl2 knockout mice. The TNF-α signaling defect is also limited to the activation of the ERK pathway. More important, whereas Tpl2+/+mice overexpressing TNF-α develop severe arthritis and inflammatory bowel disease, TNF-α-overexpressing Tpl2−/− mice develop mild forms of these syndromes. These data suggest that Tpl2 plays a critical role in both endotoxin shock syndrome and TNF-α-induced inflammation.

The data disclosed in the present invention, which were unexpected, establishes Tpl2 dependent regulation of the expression of COX-2 or Akt induced tumors. This unexpected data could not have been predicted by the prior art information on Tpl2.

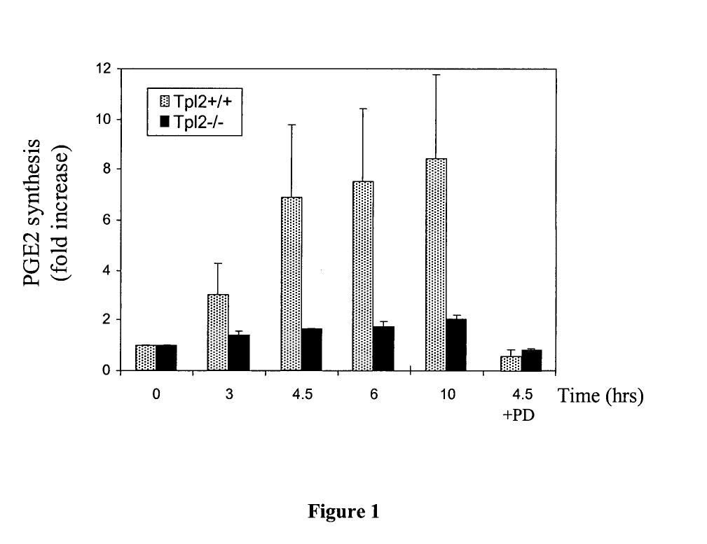

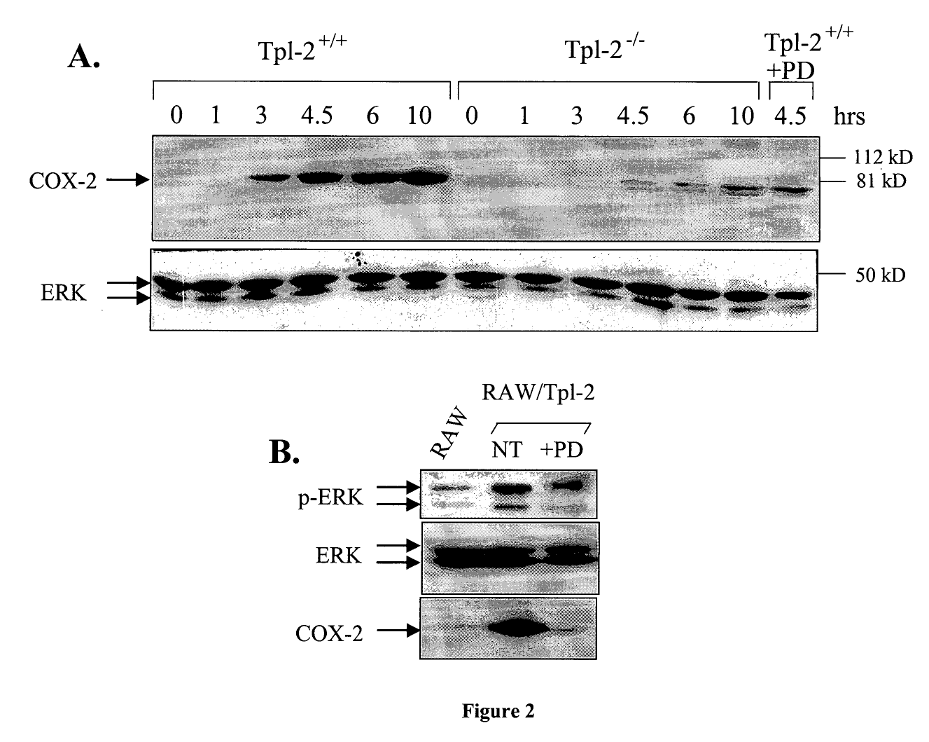

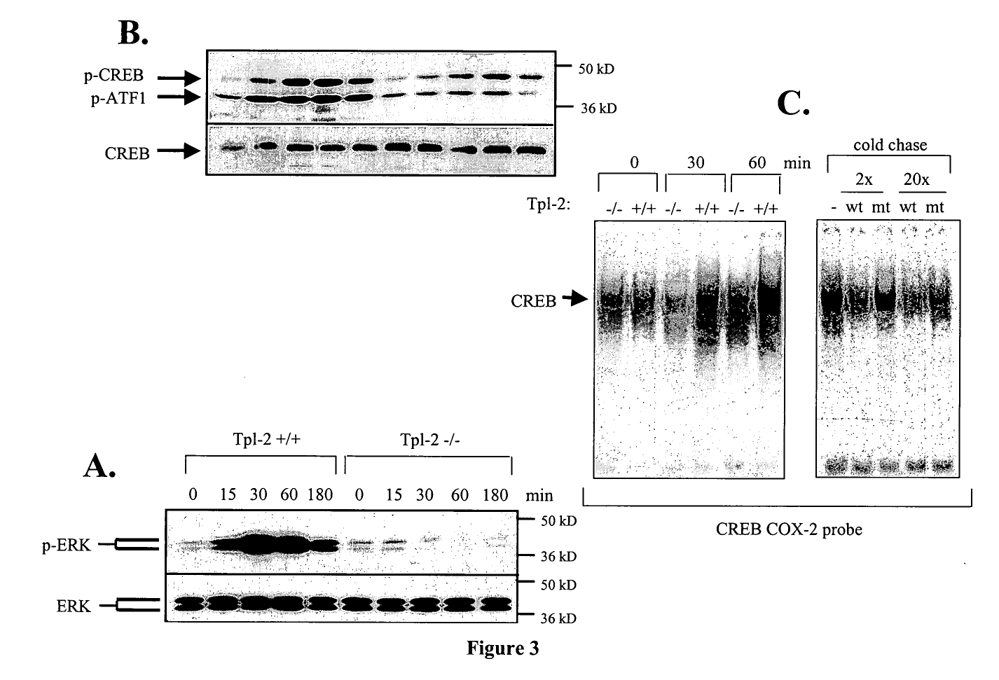

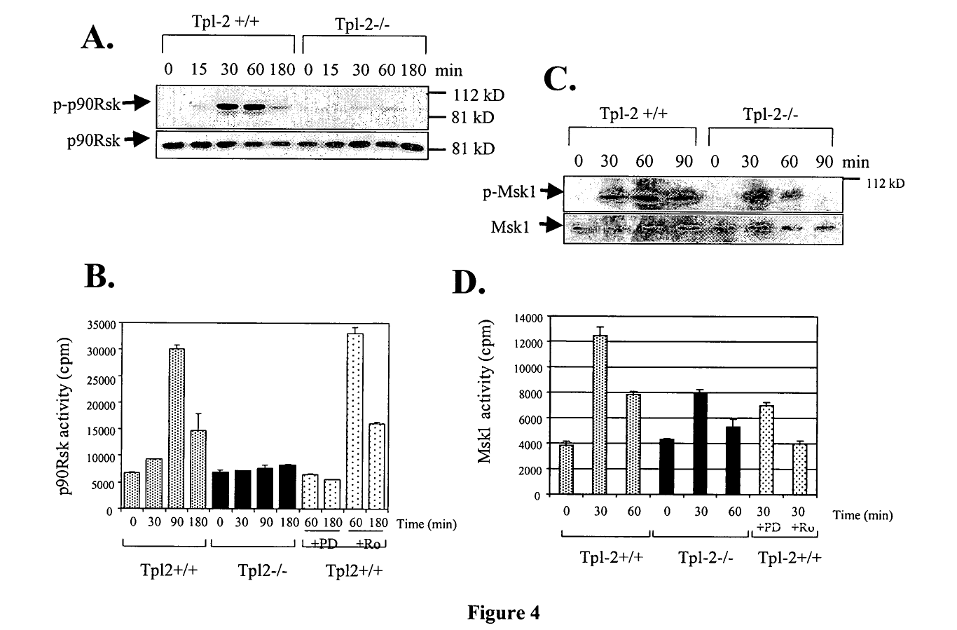

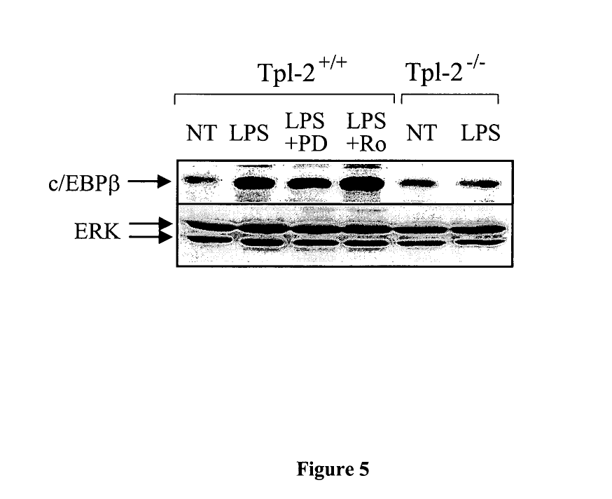

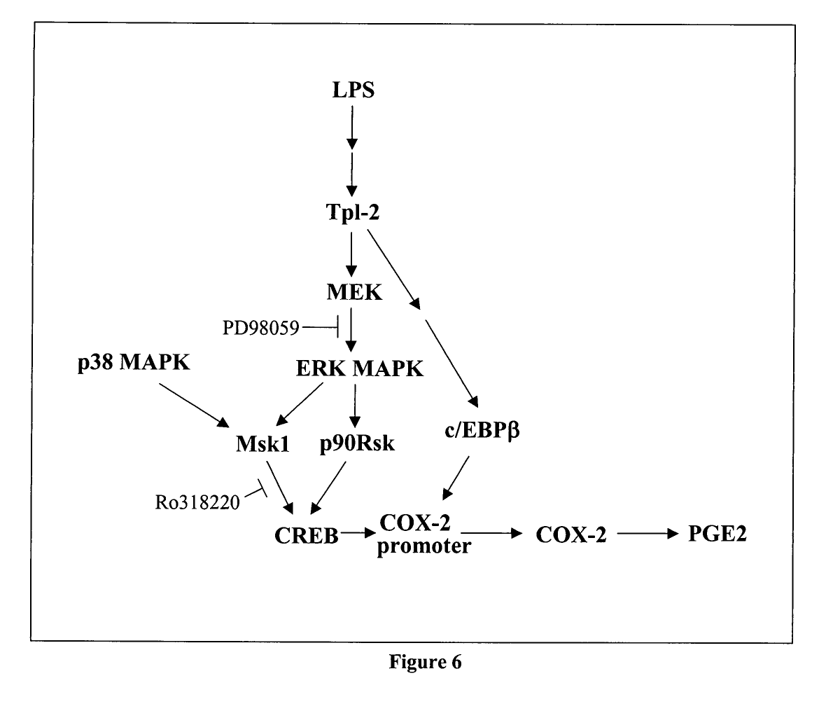

In one aspect, the present invention discloses that Tp 12 is required for the induction of cyclo-oxygenase-2 (COX-2) and prostaglandin synthesis. The data herein demonstrates that Tpl2 is required for the induction of prostaglandin synthesis in response to LPS. LPS-stimulated Tpl2−/− macrophages exhibit reduced induction of both PGE2 and its regulatory enzyme COX-2 (FIGS. 1 and 2). The defect in COX-2 induction is at the level of transcription and at the level of stability of the COX-2 message. The ability of Tpl2 to regulate COX-2 transcription was found to depend on ERK signals leading to the phosphorylation of CREB (FIG. 3) via the intermediate kinases p90Rsk and Msk1 (FIG. 4). A Tpl2-dependent but ERK-independent pathway promotes the induction of c/EBPβ which also contributes to COX-2 transactivation (FIGS. 5 and 6).

The data disclosed herein shows the role of Tpl2 in the transduction of LPS signals that regulate the expression of COX-2 and the synthesis of PGE2 in macrophages. Given the well-established role of these molecules, i.e., COX-2 and PGE2, in inflammation, the data herein clearly demonstrate that Tpl2 plays an important role in the transduction of inflammatory signals. However, both of these molecules also contribute to oncogenesis. Thus, COX-2 inhibitors decrease the incidence of colon and mammary adenocarcinomas in humans and in genetically-susceptible mice (11-17). On the opposite side, intestinal bacterial infections increase the expression of COX-2 in macrophages, and other interstitial cells in the intestines, and enhance colon tumorigenesis in Apc^{min/+} mice (18-20). In agreement with these findings, Apc^{-/+}/COX-2^{-/-} double mutant mice showed an 85% decrease in polyp induction by comparison with single APC−/+ mutants (12). Finally, Wnt-1, a known oncogene, induces COX-2 expression in mammary epithelial cell lines (21). This may contribute to the oncogenic potential of Wnt-1 because expression of COX-2 in mammary and lung carcinoma cell lines correlates with their invasive and metastatic potential (22-25). The oncogenic effects of COX-2 are prostaglandin-mediated in that the COX-2-dependent oncogenic effects of the APCΔ716 mutation are blocked in mice carrying a mutant prostaglandin receptor (EP2) gene (26).

In animals, the induction of COX-2 can be due to LPS as described above or due to other microbial or biological molecules, and/or pharmacological molecules, thereby resulting in, among other things, cancer and various inflammatory diseases including arthritis. Therefore, an approach alternative to directly targeting the expression of COX-2 in cells is to modify biochemical that interacts with COX-2, e.g., to inhibit the transduction of Tpl2 mediated signals to address the problem of cancer and inflammatory conditions. Thus, in another aspect of the invention, methods and compositions for the treatment or prevention of cancer and inflammatory conditions are provided.

The preceding data show that Tpl2 plays an important role in human cancer by regulating the metastatic potential of tumor cells, as well as by regulating angiogenesis in newly established neoplastic foci. In addition, these data suggest that Tpl2 may play an important role in the development of human cancers that are under the control of the COX-2/prostaglandin synthesis pathway. Prominent among these are tumors arising in the intestine.

In another aspect, the present invention discloses that Tpl2 is required for tumor induction by Akt. Akt (also known as PKB) was originally identified as the oncogene transduced by the acute transforming retrovirus (Akt-8) that was isolated from an AKR thymoma (27, 28, 29). Sequence analysis of the viral oncogene and its cellular homolog revealed that it encodes a serine-threonine protein kinase, composed of a carboxy-terminal kinase domain very similar to that of PKC and PKA and an amino terminal PH domain (29).

Akt/PKB is the prototype of a family of kinases that includes three known members, Akt1/PKBa (29, 30, 31) Akt-2/PKBp (32, 33), and Akt-3/PKBy (34). The Akt/PKB family of kinases is evolutionarily conserved in all eukaryotes from dictyostelium to man. The yeasts Saccharomyces cerevisiae and Saccharomyces pombe do not carry Akt, although they both carry PKC and PKA. These findings suggest that the Akt kinase family may have evolved from the PKGPKA family coincidentally with the evolution of multicellular eukaryotic species.

The three known isoforms of Akt are widely expressed, but their enzymatic activity is tightly regulated. Here we will outline the regulation of Akt-1, which has been studied best. However, evidence available to date suggests that Akt-2 and Akt-3 are repeated in a similar fashion.

Akt immunoprecipitated from unstimulated cells is catalytically inactive. However, Akt is activated rapidly by PI-3 Kinase-transduced signals induced by stimulation of a variety of receptors membrane-associated D3 phosphorylated phosphoinositides (D3PPI) induced by the activated PI-3 kinase bind the PH domain of of Akt and promote translocation of the kinase to the plasma membrane. Following translocation, Akt undergoes phosphorylation by PDKI at Thr308 and by the elusive PDK2 at Ser 473. Phosphorylation of Akt at these sites is necessary and sufficient for its activation.

Akt is a classical oncogene that was identified by viral transduction. The virus carrying this gene (Akt-8) was isolated from an AKR mouse-derived T-cell lymphoma (27) and induces thymic lymphomas when inoculated into newborn AKR mice (35). Moreover, expression of v akt, but not c-akt, into the non-oncogenic rat T-cell lymphoma line 5675 renders it highly oncogenic (36). Since v-akt contains an amino-terminal myristylation signal and exhibits different subcellular distribution than c-akt, which does not carry this signal, we proposed that it is its subcellular localization that controls its oncogenic potential. This was confirmed by experiments showing that c-akt with an amino-terminal myristylation signal derived from c-src is transforming in culture and is oncogenic when expressed in the mouse thymus or prostate. A PH domain mutant of Akt that exhibits high basal kinase activity (Akt E40K) is also oncogenic when expressed in the mouse thymus (37).

Oncogenicity studies in chickens are particularly interesting. Chickens inoculated in the wing web with a recently isolated transforming virus carrying the v-p3k oncogene encoding the catalytic subunit of PI-3K p110a develop hemangiosarcomas (38). Interestingly, a dominantnegative mutant of Akt blocks cell transformation by v-p3k. Moreover, constitutively active forms of Akt including MyrAkt, v-akt, and AktE40K induced the identical tumors in chickens, suggesting that Akt is both necessary and sufficient for the induction of hemangiosarcomas (39). Akt has also been linked to the induction of human neoplasms. The link of Akt with human oncogenesis is both direct and indirect. Direct association has been observed to date between the amplification/overexpression of Akt-2 and the development of ovarian and other epithelial neoplasms (33, 40). Indirect association has been observed in many human tumors which exhibit high Akt kinase activity in the absence of mutations directly targeting the Akt genes. Activation of Akt in these tumors results from mutations targeting signaling molecules that regulate Akt. Such mutations may target the lipid phosphatase PTEN, which has been observed both in a variety of sporadic neoplasms and in genetic syndromes predisposing to neoplasia such as Cowden's syndrome. Chronic myelogenous leukemia and acute lymphoblastic leukemia are causally linked to a t9;21 translocation that gives rise to the hybrid oncogene BCR/ABL. Recent studies showed that Akt is required for the transduction of BCR/ABL oncogenic signals that result in hematopoietic cell transformation (41). Additional indications for an indirect involvement of Akt in oncogenesis were also obtained by recent experiments showing that Akt may contribute to the transduction of angiogenic signals. Combined with the findings that Akt induces hemangiosarcomas (39), these findings suggest that Akt may contribute to the transduction of signals that promote neoplastic vascularization.

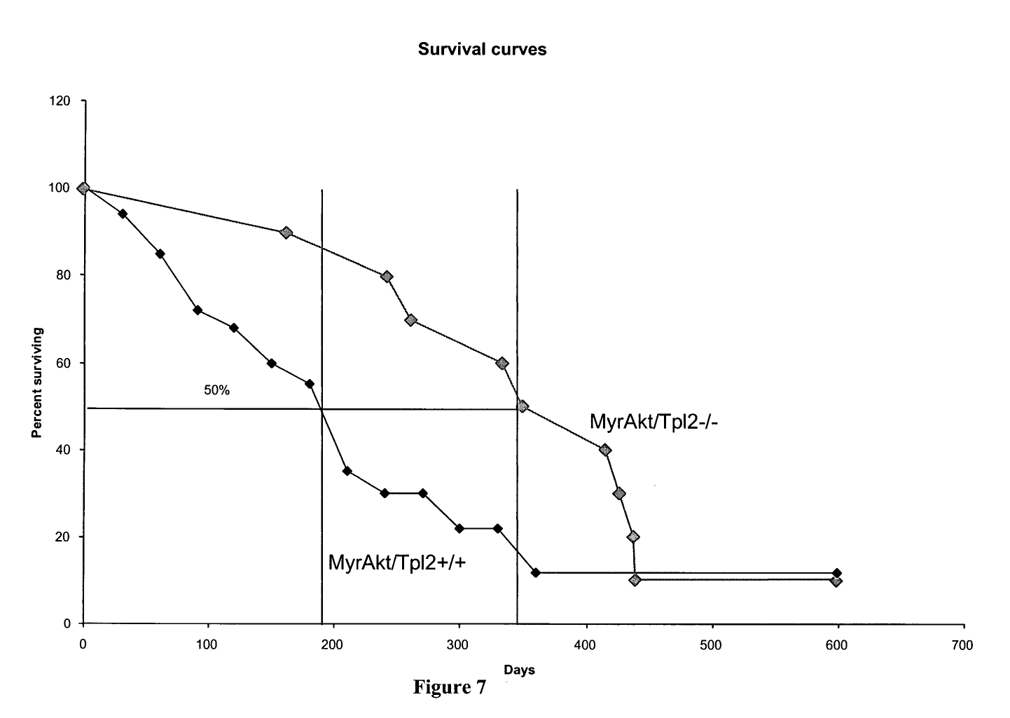

Unexpectedly, it was found in the present invention that oncogenic signals transduced via Tpl2 and Akt functionally overlap. To demonstrate this, a T-cell-directed MyrAkt transgene was placed in the Tpl2 knockout background. Observation of the aging mice over time revealed that tumor induction in MyrAkt/Tpl2−/− mice is significantly delayed by comparison with the MyrAkt/Tpl2+/+ mice (FIG. 7). This unexpected finding shows that by blocking the Tpl2 transduced signals it is possible to interfere with the oncogenic potential of Akt.

It was mentioned in the preceding paragraphs that the Akt protooncogene plays a critical role in human cancer. Inhibitors of Akt, therefore, are likely to have beneficial effects in different types of human tumors. However, it is not clear to date how serious the side effects of such inhibitors will be. The findings presented here showed that inhibition of Tpl 2 may block oncogenic signals transduced by Akt The advantage of Tpl2 inhibitors as opposed to Akt inhibitors for blocking Akt oncogenic signals lies in the fact that Tpl2 inhibitors are unlikely to be toxic. This is based on our findings showing that Tpl2 knockout mice develop normally and they are healthy.

In another aspect, the present invention also provides non-human transgenic animal models useful for screening agents useful in the modulation of cancer. The animals are genetically altered so as to express (or overexpress) a MyrAkt, preferably a human MyrAkt. The transgenic animals may be either homozygous or heterozygous for the genetic alteration. The subject animals are useful for testing candidate agents for treatment of individuals diagnosed with cancer or having propensity for developing cancer, either prophylactically or after disease onset. MyrAkt Tpl2+/+ and MyrAkt tpl2−/− mice expressing MyrAkt have been generated during the course of the present invention. See Examples section. However, before the present transgenic animals and uses therefor are described, it is to be understood that this invention is not limited to the particular methodology, protocols, cell lines, animal species or genera, constructs, and reagents described as such may, of course, vary. For example, other animal species such as rats, rabbits, pigs, goats and monkeys may also be used.

The invention is based on the discovery that blocking the Tpl 2transduced signals in transgenic mice it is possible to interfere with the oncogenic potential of Akt, thus providing an animal model for cancer treatment. The transgenic animals comprise an exogenous nucleic acid sequence present as an extrachromosomal element or stably integrated in all or a portion of its cells, especially in germ cells. The exogenous gene is usually either from a different species than the animal host, or is otherwise altered in its coding or non-coding sequence. The introduced gene may be a wild-type gene, naturally occurring polymorphism, or a genetically manipulated sequence, for example having deletions, substitutions or insertions in the coding or non-coding regions. Where the introduced gene is a coding sequence, it is usually operably linked to a promoter, which may be constitutive or inducible, and other regulatory sequences required for expression in the host animal.

In general, the transgenic animals of the invention have genetic alterations to provide for the expression of bioactive Akt. Akt nucleic acid and protein sequences are known to one skilled in the art (See, for example, GenBank numbers: NM_{—}005163 for nucleic acid sequence; NP_{—}005154 for protein:sequence; OMIM: *164730). The transgenic animals of the invention can have other genetic alterations in addition to the presence of the Akt encoding sequence. For example, the host's genome may be altered to affect the function of endogenous genes (e.g., endogenous Akt or Tpl2), contain marker genes, or other genetic alterations consistent with the goals of the present invention. For example, although not necessary to the operability of the invention, the transgenic animals described herein may have alterations to endogenous genes in addition to (or alternatively for Akt), the genetic alterations described above. For example, the host animals may be knockouts for Tpl2 as is consistent with the goals of the invention. The transgene encoding Akt should preferably provide for expression and secretion of the polypeptide as a bioactive peptide. Expression of Akt in the host animal can be either constitutive or inducible. The Akt expression may be either systemic or tissue-specific, preferably tissue-specific (e.g., thymus tissue).

How to make MyrAkt Tpl2 +/+ and MyrAkt tpl2 −/− transgenic animals are well within the grasp of one skilled in the art. For example, details such as DNA constructs, use of embryonic stem (ES) cells obtained from a host, e.g. mouse, rat, guinea pig, etc., obtaining chimeric progeny, screening the chimeric animals for the presence of the modified gene, mating the males and females having the modification to produce homozygous progeny and identifying homozygous animals are routine and one skilled in the would know how to carry out these procedures. Alternatively, tissues or organs can be maintained as allogeneic or congenic grafts or transplants, or in in vitro culture.

In drug screenig assays for modulating Tpl2 dependent COX-2 or Akt expression, cells or animals expressing these genes are used. For example, through use of the above transgenic animals or cells derived therefrom, one can identify ligands or substrates that modulate the interaction of Tpl-2 and COX-2 or Tpl-2 and Akt and the phenomena associated with the interaction, such as cancer. Of particular interest are screening assays for agents that have a low toxicity for human cells. A wide variety of assays may be used for this purpose, and depending on the particular assay, whole animals may be used, or cells derived therefrom. Cells may be freshly isolated from an animal, or may be immortalized in culture. Cells of particular interest are derived from thymus tissue or cancerous tissue. Any molecule, e.g. protein or pharmaceutical, predicted to have the capability of affecting the molecular and clinical phenomena associated with the interaction of Tpl-2 and COX-2 or Tpl-2 and Akt can be used. Generally a plurality of assay mixtures are run in parallel with different agent concentrations to obtain a differential response to the various concentrations. Typically, one of these concentrations serves as a negative control, i.e. at zero concentration or below the level of detection.

Candidate agents can be, for example, organic molecules, preferably small organic compounds or other classes of molecules or compounds. Candidate agents are obtained from a wide variety of sources including libraries of synthetic or natural compounds. For example, detection may utilize staining of cells or histological sections, performed in accordance with conventional methods. The antibodies of interest are added to the cell sample, and incubated for a period of time sufficient to allow binding to the epitope. Such reagents are well known in the art. A number of assays are known in the art for determining the effect of a drug on cancer. The subject animals may be used by themselves, or in combination with control animals. Preferably, the screen will include control values (e.g., the level of Akt expression or production in the test animal in the absence of test compound(s)). Test substances which are considered positive, i.e., likely to be beneficial in the treatment of cancer or inflammation, will be those which have a substantial effect. (e.g., test agents that are able to reduce the level of Akt or COX-2 production, preferably by at least 20% more preferably by at least 50%, and most preferably by at least 80%, still more preferably by about 90%.

For example, a method for identifying a compound or agent in its simplest form may include incubating a candidate compound or compounds to be tested with a Tpl-2 molecule and COX-2 or Tpl-2 and Akt molecules, under conditions in which, but for the presence of the compound or compounds to be tested, the interaction of Tpl-2 and COX-2 or Tpl-2 and Akt induces a detectable or measurable biological effect or a chemical effect and then determining the ability of COX-2 or Akt to interact with Tpl-2 to induce a detectable or measurable biological effect or a chemical effect in the presence of the compound or compounds to be tested. If the candidate compound or the tested compound modulates the interaction of COX-2 or Akt to interact with Tpl-2, then that compound is selected.

### EXAMPLES

The following examples further illustrate the present invention. The examples below are carried out using standard techniques, that are well known and routine to those of skill in the art, except where otherwise described in detail. The examples are illustrative and do not limit the invention.

### Example 1

### PGE2 Levels in LPS Treated Macrophages

Tpl2+/+ and Tpl2−/− macrophage cultures were stimulated with 1 fig/ml LPS. Culture supernatants collected at 0, 3, 4.5, 6 or 10 hrs were analysed for PGE2 levels by ELISA. Unstimulated Tpl2+/+ and Tpl2−/− macrophage cultures secreted similar levels of PGE2 respectively. PGE2 levels produced by unstimulated cells were given the arbitrary value of 1. The bar graphs show the fold induction of PGE2 (±SD) compared to control untreated cultures. Pre-treatment with 20 pM of the MEK inhibitor PD98059 inhibited PGE2 secretion in Tpl2+/+ macrophages stimulated for 4.5 hrs with LPS. As shown in FIG. 1, LPS-treated Tpl2−/− macrophages secrete reduced levels of PGE2.

### Example 2

### Induction of COX-2 in LPS-Stimulated Macrophages

Induction of COX-2 in LPS-stimulated macrophages were studied and the data is illustrated in FIG. 2: Specifically, as shown in FIG. 2(A) COX-2 induction by LPS depends on signals transfuced via Tpl2. Lysates from Tpl2+/+(lanes 1-6) and Tpl2−/− (lanes 7-12) macrophages were treated with 1 μg/ml LPS and they were harvested at the indicated time points. Western blot of cell lysates was probed with an antibody against COX-2 (upper panel). To assess the contribution of ERK in the Tpl2-dependent induction of COX-2, Tpl2+/+ macrophages were pre-treated with 20 pM of PD98059 for 45 min and then stimulated for 4.5 hrs with LPS (last lane, upper panel). Immunoblotting with an anti-ERK antibody was used to determine gel loading and transfer efficiency (lower panel). As shown in FIG. 2(B), Exogenous Tpl2 expression in RAW264.7 macrophages activates ERK and induces the expression of COX-2. Untransfected RAW264.7 cells and RAW264.7 cells stably transfected with a Tpl2 expression construct were cultured with or without the MEK inhibitor PD98059 (20 pm for 12 h). Western blots of lysates derived from these cells were probed with an antibody that recognizes phosphorylated ERKI and ERK2 (upper panel) or total ERKI and ERK2 (middle panel). To determine whether Tpl2-induced ERK activation is sufficient to induce COX-2 expression, the same blot was probed with an antibody against COX-2 (lower panel). Thus, it is shown here that Tpl2 is required for optimal induction of COX-2 in LPS-stimulated macrophages.

### Example 3

### Phosphorylation and DNA Binding Activity of CREB in LPS-Stimulated Macrophages

Phosphorylation and DNA binding activity of CREB in LPS-stimulated macrophages were studied and the data is presented in FIG. 3: Specifically, in FIG. 3 (A), data related to kinetics of ERK phosphorylation in cell lysates from Tpl2+/+ (lanes 1-5) and Tpl2−/(lanes 6-10) macrophages stimulated with LPS (1 μg/ml) are shown. Western blot of cell lysates harvested at the indicated time points were probed with an antibody against the phosphorylated forms of p44 ERKI and p42 ERK2 (upper panel) or with an antibody that detects total ERK (lower panel). In FIG. 3(B), data relating to kinetics of CREB/ATFI phosphorylation in cell lysates from Tpl2+/+ (lanes 1-5) and Tpl2−/−(lanes 6-10) macrophages stimulated with LPS (I μg/ml) are shown. Western blots of cell lysates harvested at the indicated time points was probed with an antibody against CREB and ATF1 phosphorylated at Ser133 and Ser63, respectively (upper panel) or with an antibody which detects total CREB (lower panel).

In FIG. 3(C), it is shown that the induction of CREB DNA binding activity by LPS is Tpl2-dependent. Left panel. Nuclear extracts of primary Tpl2+/+ (lanes 2, 4 and 6) and Tpl2−/− macrophages (lanes 1, 3 and 5) treated with LPS (I μg/ml) for 0, 30 min or 60 min were analysed by EMSA for binding to a ‘²P-labeled synthetic oligonucleotide containing the CREB binding motif of the COX-2 promoter. Right panel. Nuclear extracts from Tpl2+/+ cells treated with LPS for 1 hour, were incubated with excess (2-fold or 20-fold) unlabelled wild type (wt) or mutant (mt) probe prior to being analysed for CREB-binding activity by EMSA. In sum, it is shown here that Tpl2 regulates the phosphorylation and DNA binding activity of CREB in LPSstimulated macrophages.

### Example 4

### Phosphorylation and Activation of the Protein Kinases p90Rsk and MskL

The data related to the phosphorylation and activation of the protein kinases p90Rsk and Msk has been shown in FIG. 4. Specifically, in FIG. 4(A), it is shown that the phosphorylation of p90Rsk in LPS-stimulated Tpl2−/− macrophages is impaired. Western blots of cell lysates from LPS-stimulated (1 μg/ml) Tpl2+/+(lanes 1-5) and Tpl2−/− (lanes 6-10) macrophages, harvested at the indicated time points, were probed with antibodies specific for the phosphorylated form of p90Rsk (upper panel) or for total p90Rsk (lower panel). In FIG. 4(B), it is shown that the activation of p90Rsk by LPS in Tpl2−/− macrophages is impaired. P90RSK kinase activity was measured in lysates of LPS-stimulated Tpl2+/+ and Tpl2−/− macrophages, or Tpl2+/+ macrophages pre-treated with PD98059 or Ro318220 and harvested at the indicated time points. The synthetic peptide AKRRRLSSLRA was used as the substrate. Kinase activity is expressed in cpms of ³²P-ATP incorporated by p90RSK immunoprdcipitated from 0.25 mg of lysate. The values shown are mean values from triplicate determinations in a representative experiment. p90Rsk kinase assays were repeated in four independent experiments and gave similar results. In FIG. 4(C), it is shown that the phosphorylation of Msk1 in LPS-stimulated Tpl2−/− macrophages is impaired. Western blots of lysates from LPS-stimulated (1 μg/ml) Tpl2+/+ (lanes 1-4) and Tpl2−/− (lanes 5-8) macrophages harvested at the indicated time points, were probed with antibodies specific for the phosphorylated form of Msk1 (upper panel) or for total Msk1 (data not shown). In FIG. 4(D), it is shown that the activation of Msk1 by LPS in Tpl2 I-macrophages is impaired. Endogenous Msk1 was immunoprecipitated from lysates of LPS-stimulated Tpl2+/+ and Tpl2−/− macrophages or Tpl2+/+ macrophages pre-treated with PD98059 or Ro318220, and harvested at the indicated time points. In vitro kinase assays were carried out on the immunoprecipitates using the synthetic peptide EILSRRPSYRK (CREBtide) as substrate. Kinase activity is expressed in cpms of ³²P-ATP incorporated by Msk1 immunoprecipitated from 1 mg of lysate. The values shown are mean values from triplicate determinations in a representative experiment. Msk1 kinase assays were repeated in three independent experiments and gave similar results. Thus, it has been demonstrated here that Tpl2 transduces LPS signals leading to the phosphorylation and activation of the protein kinases p90Rsk and MskL

### Example 5

### Expression of C/EBPβ

In FIG. 5 the data relating to the expression of C/EBP□ has been shown. To demonstrate this, a Western blot of cell lysates derived from unstimulated and LPS-stimulated (1 μg/ml for 6 h) Tpl2+/+ and Tpl2−/− macrophages were probed with an antibody specific for C/EBP□ or ERK1 and ERK2. Some of the cultures were treated with PD98059 (20 μm) or Ro318220 (3 μM) for 45 min prior to their exposure to LPS. Data shown are representative of 3 independent experiments. Thus, it has been shown here that LPS induces the expression of C/EBP [symbol] via a Tpl2-dependent, ERK and Msk1 independent pathway.

A model of Tpl 2-mediated COX-2 transactivation and PGE2 production in response to LPS is shown in FIG. 6. LPS engages a Tpl2-dependent pathway which leads to the activation of ERK downstream of MEK. ERK-transduced signals activate p90Rsk. The same signals, in combination with p38 MAPK-transduced signals activate Msk1. p90Rsk and Msk1, in turn, phosphorylate and activate CREB. CREB phosphorylation and activation is critical for LPS-induced transactivation of COX-2, a key enzyme for the biosynthesis of PGE2. A complementary pathway which is Tpl2dependent but ERK-independent, is obligatory for the induction of C/EBPβ, a transcription factor known to contribute to the induction of COX-2. The interrupted line connecting Tpl2 with p38MAPK indicates that signals transduced via Tpl2 may contribute to the activation of p38MAPK by LPS. However, these signals are not obligatory for p38MAPK activation. The sites of action of the kinase inhibitors PD98059 and Ro318220 are also indicated.

### Example 6

### Generation of MyrAkt Tpl2+/+ and MyrAkt tpl2 −/− Mouse Models and Tumor Induction by MyrAkt in These Models

Generation and Establishment of Tpl-2 −/− mice (also referred to as Tpl-2 null mice or knockout mice herein) were carried out as decribed in WO 01/66559, the contents of which are incorporated herein by reference.

The MyrAkt1 mice, expressing the active form of Akt in the T cells, were previously reported (Malsrom et al., 2001, PNAS, 98(26): 14967-14972). These transgenic mice develop thymomas and die at a median age of 190 days. They contained a MyrAkt transgene under the control of a T-cell specific Lck promoter 10 which ensures that the transgene is expressed in T cells.

For the generation of transgenic Tpl2−/− mice expressing the Akt, the mice expressing Lck-MyrAkt males were bred to Tpl2 −/− mice. The transgenic mice from the first generation (F1), all of which were Tpl2 +/−, were crossed again to Tpl2 −/− mice. From the transgenic mice of the second generation (F2), the Tpl2 −/− mice expressing the Akt were selected. These mice developed tumors and died at a median age of 350 days.

In FIG. 7 data related to tumor induction by MyrAkt in vivo using the mouse model is shown. MyrAkt Tpl2 +/+ and MyrAkt tpl2 −/− mice expressing MyrAkt in the thymus were aged and scored for tumor induction. The results showed tumor induction is significantly delayed in MyrAkt transgenic mice lacking Tpl2. Therefore, Tpl2 inactivation protects or at least delays from tumor induction by MyrAkt.

### LIST OF REFERENCE CITED

1) Patriotis, C., Makris, A., Bear, S. E. and Tsichlis, P. N. (1993) Tumor progression locus 2 (Tpl-2) encodes a protein kinase involved in the progression of rodent T-cell lymphomas and in T-cell activation. Proc Natl Acad Sci USA, 90,2251-5.

2) Emy, K. M., Peli, J., Lambert, J. F., Muller, V. and Diggelmann, H. (1996) Involvement of the Tpl-2/cot oncogene in MMTV tumorigenesis. Oncogene, 13, 2015-20.

3) Patriotis, C., Makris, A., Chemoff, J. and Tsichlis, P. N. (1994) Tpl-2 acts in concert with Ras and Raf-1 to activate mitogen-activated protein kinase. Proc. Natl. Acad. Sci. USA, 91, 9755-9759.

4) Ceci, J. D., Patriotis, C. P., Tsatsanis, C., Makris, A. M., Kovatch, R., Swing, D A., Jenkins, N. A., Tsichlis, P. N. and Copeland, N. G. (1997) Tpl-2 is an oncogenic kinase that is activated by carboxy-terminal truncation. Genes Dev., 11, 688-700.

5) Belich, M. P., Salmeron, A., Johnston, L. H. and Ley, S. C. (1999) Tpl-2 kinase regulates the proteolysis of the NF-xB-inhibitory protein NF-xB1 p105. Nature, 397, 363-368.

6) Lin, X., Cunningham, E. T., Mu, Y., Geleziunas, R. and Greene, W. C. (1999) The protooncogene Cot kinase participates in CD3/CD28 induction of NF-xB acting through the NF-xB-inducing kinase and IxB kinases. Immunity, 10, 271-280.

7) Salmeron, A., Ahmad, T. B., Carlile, G. W., Pappin, D., Narsimhan, R. P. and Ley, S. C. (1996) Activation of MEK-1 and SEK-1 by Tpl-2 proto-oncoprotein, a novel MAP kinase kinase kinase. EMBO J, 15, 817-826.

8) Tsatsanis, C., Patriotis, C., Bear, S. E. and Tsichlis, P. N. (1998) The Tpl-2 protooncoprotein activates the nuclear factor of activated T cells and induces interleukin 2 expression in T cells. Proc. Natl. Acad. Sci. USA, 95, 3827-3832.

9) Tsatsanis, C., Patriotis, C. and Tsichlis, P. N. (1998b) Tpl-2 induces IL-2 expression in T cell lines by triggering multiple signaling pathways that activate NFAT and NF-xB. Oncogene, 17, 2609-2618.

10) Dumitru, C. D., Ceci, J. D., Tsatsanis, C., Kontoyiannis, D., Stamatakis, K., Lin, J. H., Patriotis, C., Jenkins, N. A., Copeland, N. G., Kollias, G. and Tsichlis, P. N. (2000) TNF-a induction by LPS is regulated posttranscriptionally via a Tpl2/ERK-dependent pathway. Cell, 103, 1071-83.

11) DuBois R N, Giardiello F M, Smalley W E. (1996) Nonsteroidal anti-inflammatory drugs, eicosanoids, and colorectal cancer prevention. Gastroenterol Clin North Am. 25:773-91.

12) Oshima, M., Dinchuk, J. E., Kargman, S. L., Oshima, H., Hancock, B., Kwong, E., Trzaskos, J. M., Evans, J. F. and Taketo, M. M. (1996) Suppression of intestinal polyposis in Apc delta716 knockout mice by inhibition of cyclooxygenase 2 (COX-2). Cell, 87, 803-9.

13) Elder, D. J. and Paraskeva, C. (1998) COX-2 inhibitors for colorectal cancer. Nat Med, 4, 392-3

14) Jacoby, R. F., Seibert, K., Cole, C. E., Kelloff, G. and Lubet, R. A. (2000) The cyclooxygenase-2 inhibitor celecoxib is a potent preventive and therapeutic agent in the min mouse model of adenomatous polyposis. Cancer Res, 60, 5040-4.

15) Jones, M. K., Wang, H., Peskar, B. M., Levin, E., Itani, R. M., Sarfeh, I. J. and Tamawski, A. S. (1999) Inhibition of angiogenesis by nonsteroidal anti-inflammatory drugs: insight into mechanisms and implications for cancer growth and ulcer healing. Nat Med, 5, 141823.

16) Oshima, M., Murai, N., Kargman, S., Arguello, M., Luk, P., Kwong, E., Taketo, M. M. and Evans, J. F. (2001) Chemoprevention of intestinal polyposis in the Apcdelta716 mouse by rofecoxib, a specific cyclooxygenase-2 inhibitor. Cancer Res, 61, 1733-40. 17)

17) Rozic, J. G., Chakraborty, C. and Lala, P. K. (2001) Cyclooxygenase inhibitors retard murine mammary tumor progression by reducing tumor cell migration, invasiveness and angiogenesis. Int J Cancer, 93, 497-506.

18) Hull, M. A., Booth, J. K., Tisbury, A., Scott, N., Bonifer, C., Markham, A. F. and Coletta, P. L. (1999) Cyclooxygenase 2 is up-regulated and localized to macrophages in the intestine of Min mice. Br J Cancer, 79, 1399-405.

19) Williams, C. S., Luongo, C., Radhika, A., Zhang, T., Lamps, L. W., Nanney, L. B., Beauchamp, R. D. and DuBois, R. N. (1996) Elevated cyclooxygenase-2 levels in Min mouse adenomas. Gastroenterology, 111, 1134-40

20) Newman J V, Kosaka T, Sheppard B J, Fox J G, Schauer D B. (2001) Bacterial infection promotes colon tumorigenesis in Apc(Min/+) mice. J Infect Dis. 184:227-30.

21) Howe L P, Subbaramaiah K, Chung W J, Dannenberg A J, Brown A M. (1999) Transcriptional activation of cyclooxygenase-2 in Wnt-1-transformed mouse mammary epithelial cells. Cancer Res. 59:1572-7.

22) Hida, T., Yatabe, Y., Achiwa, H., Muramatsu, H., Kozaki, K., Nakamura, S., Ogawa, M., Mitsudomi, T., Sugiura, T. and Takahashi, T. (1998) Increased expression of cyclooxygenase 2 occurs frequently in human lung cancers, specifically in adenocarcinomas. Cancer Res, 58, 3761-4.

23) Kundu N, Yang Q, Dorsey R, Fulton A M. (2001) Increased cyclooxygenase-2 (cox-2) expression and activity in a munne model of metastatic breast cancer. Int J Cancer. 93:681-6.

24) Kozald K, Koshikawa K, Tatematsu Y, Miyaishi O, Saito H, Hida T, Osada H, Takahashi T. (2001) Multi-faceted analyses of a highly metastatic human lung cancer cell line NCI-H460-LNM35 suμgest mimicry of inflammatory cells in metastasis. Oncogene. 20:422834.

25) Liu, C. H., Chang, S. H., Narko, K., Trifan, O. C., Wu, M. T., Smith, E., Haudenschild, C., Lane, T. F. and Hla, T. (2001) Overexpression of cyclooxygenase-2 is sufficient to induce tumorigenesis in transgenic mice. J Biol Chem, 276, 18563-9.

26) Sonoshita, M., Takaku, K., Sasaki, N., Sugimoto, Y., Ushikubi, F., Narumiya, S., Oshima, M. and Taketo, M. M. (2001) Acceleration of intestinal polyposis through prostaglandin receptor EP2 in Apc(Delta 716) knockout mice. Nat Med, 7, 1048-51.

27) Staal S P, Hartley J W, Rowe W P. 1977. Proc. Natl. Acad. Sci. USA 74:3065-67 28) Staal S P. 1987. Proc. Natl. Acad. Sci. USA 84:5034-37

29) Bellacosa A, Testa J R, Staal S P, Tsichlis P N. 1991. Science 254:274-77

30) Coffer P J, Woodgett J R. 1991. Eur. J. Biochem. 201:475-81

31) Jones P F, Jakubowicz T, Pitossi F J, Maurer F, Hemmings B A. 1991. Proc. Natl. Acad. Sci. USA 88:4171-75

32) Jones P F, Jakubowicz T, Hemmings B A. 1991. Cell Regul. 2:1001-9.

33) Cheng J Q, Godwin A K, Bellacosa A, Taguchi T, Franke T F, et al. 1992. Proc. Natl. Acad. Sci. USA. 89:9267-71

34) Konishi H, Kuroda S, Tanaka M, Mat-suzaki H, Ono Y, et al. 1995. Biochem. Biophys. Res. Commun. 216:526-34

35) Staal S P, Hartley J W. 1988. J. Exp. Med. 167:1259-64

36) Ahmed N N, Franke T F, Bellacosa A, Datta K, Gonzalez P M, et al. 1993. Oncogene 8:1957-63

37) Malstrom S, Tili E, Kappes D, Ceci J D, Tsichlis P N. Tumor induction by an Lck-MyrAkt transgene is delayed by mechanisms controlling the size of the thymus. Proc Natl Acad Sci USA 2001 Dec. 18;98(26):14967-72

38) Chang H W, Aoki M, Fruman D, Auger K R, Bellacosa A, et al. 1997. Science 276:1848-50

39) Aoki M, Batista O, Bellacosa A, Tsichlis P, Vogt P K. 1998. Proc. Natl. Acad. Sci. USA 95:14950-55

40) Bellacosa A, de Feo D, Godwin A K, Bell D W, Cheng J Q, et al. 1995. Int. J. Cancer 64:280-85

41). Skorski T, Bellacosa A, Nieborowska-Skorska M, Majewski M, Martinez R, et al. 1997. EMBO J. 16:6151-61

All publications, patents and patent applications mentioned in this specification are indicative of the level of those skilled in the art to which this invention pertains. The contents of all the publications, patents and patent applications are incorporated herein by reference to the same extent as if each individual publication, patent or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method of selecting a compound for treatment of cancer or inflammation, wherein the cancer or inflammation is under the control of COX-2 expression/prostaglandin synthesis pathway, the method comprising:
evaluating the anti-Tpl2 activity of the compound;
evaluating whether the compound down-regulates the COX-2 expression; and
selecting the compound that exhibits anti-Tpl2 activity and down regulates the COX-2 expression.

2. The method of claim 1, wherein the step of evaluating whether the compound down-regulates the COX-2 expression comprises testing for phosphorylation and DNA binding activity of CREB.

3. The method of claim 1, wherein the step of evaluating whether the compound down-regulates the COX-2 expression comprises testing for phosphorylation and activation of a protein kinase selected from the group consisting of p90Rsk and Msk1.

3. The method of claim 1, wherein the steps of evaluation is done either in vitro cultured cells or in an animal model.

4. The method of claim 3, wherein the step of evaluating whether the compound down-regulates the COX-2 expression comprises testing for activity of a COX-2 promter-luciferase reporter gene in the cells.

5. The method of claim 1, wherein the animal model is selected from the group consisting of a rat, a rabbit, a pig, a cow, a monkey and a guinea pig.

6. A method for identifying a compound with potential for treating neoplasia or inflammation, comprising:
evaluating a compound known to have an anti-Tpl2 activity for its ability to down-regulates the COX-2 expression; and
selecting the compound that down regulates the COX-2 expression.

7. A method of selecting a compound for treatment of Akt induced tumor growth, the method comprising:
evaluating the anti-Tpl2 activity of the compound;
evaluating whether the compound blocks an interaction between Tpl-2 and Akt; and
selecting the compound that interaction between Tpl-2 and Akt.

8. The method of claim 7, wherein the steps of evaluation is done either in vitro cultured cells or in an animal model.

9. The method of claim 8, wherein the animal model is selected from the group consisting of a rat, a rabbit, a pig, a cow, a monkey or a guinea pig.

10. A transgenic Tpl2−/− mouse whose genome comprises a nucleic acid sequence encoding an Akt protooncogene operatively linked to a promoter, wherein the mouse is characterized by its ability to show delayed tumor induction by comparison with a transgenic Tpl2+/+ mouse expressing the Akt.

11. A composition comprising macrophages isolated from the transgenic Tpl2−/− mouse of claim 10.

12. A method of screening for biologically active compounds that modulate tumor induction associated with the interaction of Tpl-2 and Akt, the method comprising:
administering or combining a candidate compound to a transgenic Tpl2+/+ mouse and Tpl2−/−mouse, both comprising a transgenic nucleotide sequence encoding bioactive Akt operably linked to a promoter and stably integrated into the genome of each mouse, wherein said nucleotide sequence is expressed and wherein said expression results in delayed tumor induction in the transgenic Tpl2−/−mouse; and
determining the effect of the compound upon tumor induction in the transgenic mice; and
selecting the compound that controls or delays tumor induction in the Tpl2+/+ mouse as seen in the Tpl2−/−mouse.

13. The metod of claim 12, wherein the compound is administered orally, parenterally, subcutaneously, intramuscularly or intravascularly or topically.

14. A method of screening for biologically active compounds that modulate a pathology associated with cancer, wherein the parthology is promoted by the interaction of Tpl-2 and Akt, the method comprising:
administering a candidate compound to a transgenic mouse comprising a transgenic nucleotide sequence encoding bioactive Akt operably linked to a promoter and stably integrated into the genome of the mouse, wherein said nucleotide sequence is expressed and wherein said expression results in tumor induction; and
determining the effect of the compound upon the pathology promoted by the interaction of Tpl-2 and Akt.

15. A method of treating an individual suffering from an Akt induced cancer comprising administering to the individual a therapeutically effective amount of an inhibitor or antagonist of Tpl-2, wherein the inhibitor or antagonist blcoks Akt induced tumors.

16. The method of claim 15, wherein the inhibitor or antagonist of Tpl-2 is a peptide, a mutant Tpl2 protein, nucleic acid, an organic compound or a small molecule targeted to Tpl2.
